(19) United States
(12) Patent Application Publication
(10) Pub. No.: US 2007/0038275 A1
(43) Pub. Date: Feb. 15, 2007

(54) High-frequency electrotherapy apparatus

(76) Inventor: Hee-Je Kim, Geumieong-Ku (KR)

Correspondence Address: KNOBBE MARTENS OLSON & BEAR LLP, 2040 MAIN STREET, FOURTEENTH FLOOR, IRVINE, CA, 92614, US

(21) Appl. No.: 11/480,611

(22) Filed: Jun. 30, 2006

(30) Foreign Application Priority Data
Jul. 5, 2005 (KR) 10-2005-0060430

(51) Int. Cl. A61N 5/06

(52) U.S. Cl. 607/90; 607/154; 607/66

(57) ABSTRACT

A high-frequency electrotherapy apparatus capable of controlling a variety of output, frequency, and pulse shape is disclosed. The high-frequency electrotherapy apparatus comprises a main body having a switching controller controlling through a digital signal processor (DSP) and a display controller controlling through a microcontroller, and an infrared lamp for the optimum hair-growth effect. High-frequency current is transmitted through a human body using a treatment electrode contacted with a scalp and a counter electrode pad for coupling with the treatment electrode. In the high-frequency electrotherapy apparatus according to the present invention, all the conditions such as the output, frequency, and pulse shape are configured to accomplish the optimum effect for growth of hair. Also, dangerous factors for the human body are removed using a feedback circuit and a grounded frame of the main body, thereby prevent burning or electric shock. Moreover, infiltration depth of the high-frequency is minimized to approximately 2mm, so that diathermy can be achieved without inducing any side effect of the electrical therapy.

### CROSS-REFERENCE TO RELATED APPLICATIONS

This application claims the benefit of Korean Patent Application No. 10-2005-0060430, filed on Jul. 5, 2005 in the Korean Intellectual Property Office, which is incorporated herein by reference in its entirety.

### BACKGROUND OF THE INVENTION

1. Field of the Invention

The present invention relates to a high-frequency electrotherapy apparatus. More particularly, the present invention relates to a high-frequency electrotherapy apparatus capable of maximizing effect on growth of hair by removing harmful environmental factors.

2. Description of the Related Technology

Generally, electrotherapy refers to a medical treatment that utilizes electricity such as direct current, alternating current, and pulsation current to diagnose and treat diseases. The electrotherapy includes a medical galvanism, iontophoresis, electrical stimulation therapy, transcutaneous electrical nerve stimulation (TENS) therapy, functional electrical stimulation (FES) therapy, interferential current therapy (ICT), shortwave diathermy (SWD) therapy, microwave diathermy (MWD) therapy, ultrasonic therapy, and the likes.

The electrotherapy can be used, for example, to treat damages and ailments in a musculoskeletal system, a nervous system and a circulating system, and to treat dermatitis, internal diseases, and chronic inflammation. Such electrotherapy is advantageous in that the ailments and diseases can be treated using physical phenomena and characteristics, by inducing electrical changes in a human body through electrical stimulation from the outside.

In general, the functions and effects of the electrotherapy vary according to the frequency being applied, the peak value of voltage, the type and strength of electric current, and the body part that is treated. Other factors, such as the structure, size and material of a pad applied to a human body, application time, and use of antibiotics or other medications can also influence the effects.

Capacitive and resistive electric transfer generates various somatological effects. For example, when body temperature is increased by applying the electric transfer, blood vessels expand, thereby vitalizing organizations and promoting blood circulation. Also, oxygenatation and catabolin excretion are stimulated.

An electrotherapy apparatus is generally an output device utilizing the electric transfer therapy to apply electrical stimulation onto specific body parts, such as the waist, stomach, legs, shoulders and soles of the feet. A plurality of pads can be provided so that the electrical stimulation can be transmitted through the pads attached to the body parts, by supplying signals having the same frequency characteristics.

In an electrotherapy apparatus, however, effects of the stimulation by the signal may not be sufficient. Furthermore, since the pads need to be handled by experts, in applying adhesive onto a frequency input terminal or in using a belt, a domestic use of the electrotherapy apparatus is limited.

An electrotherapy apparatus having a pad-type input terminal may cause a burn and electric shock to a user. Moreover, the effects of therapy may not be satisfactory because the frequency of the current generated during the electrotherapy is irregular.

### SUMMARY OF CERTAIN INVENTIVE ASPECTS

One aspect of the invention provides a high-frequency electrotherapy apparatus capable of providing effective therapy without causing significant bodily harm. The high-frequency electrotherapy apparatus comprises: a main body comprising: a current generator for generating a high frequency current; a control unit for controlling generation of a high frequency current, and a grounded frame; a treatment electrode electrically connected to the main body, the treatment electrode being configured to contact a body part of a patient; a counter electrode pad electrically connected to the main body, the counter electrode being configured to contact another body part of the patient; and an infrared lamp electrically connected to the main body.

The high-frequency current may have a voltage of about 1.0 to about 1.5 kV, a frequency of about 250 to about 330 kHz, and an average input current of about 0.2 to about 0.7 A. The main body may further comprise: a rectification unit for rectifying power supply; an output control unit connected to an output of the rectification unit for controlling a current output from the rectification unit; a stabilizing circuit unit connected to an output of the output control unit for blocking abnormal signals when switching; a transform unit having an input connected to an output of the stabilizing circuit unit and an output connected to a load, the transform unit being configured to boost a voltage applied to the load; an intermittence unit for switchingly controlling a waveform of the voltage output from the transform unit; and a feedback circuit unit for preventing a current above a reference value.

The control unit may comprise: a switching controller for measuring and controlling through a digital signal processor (DSP); and a display controller for controlling setting of output voltage and output energy, input of run/stop commands, cover-current protection, and display, through a microcontroller. When current above the reference value is detected by the feedback circuit unit, the control unit may interrupt application of a signal and current to the intermittence unit.

The treatment electrode may comprise: a cylindrical shaft; a terminal connected to the shaft; and a thin ceramic layer coating the terminal. A skin-contact area of the counter electrode pad is approximately 20 times as large as a skin-contact area of the treatment electrode pad. The intermittence unit may switchingly control a waveform of the voltage output from the transform unit to have an optimum pulse shape for maximizing a hair growth effect. The transform unit may be configured to adjust inductance so as to remove a ripple voltage generated between the electrodes. The infrared lamp may be configured to project infrared rays onto the body part.

Another aspect of the invention provides a high-frequency electrotherapy apparatus comprising: a main body comprising: a grounded frame, a rectification unit for rectifying power supply, an output control part connected to an output of the rectification unit for controlling a current output from the rectification unit, a stabilizing circuit unit connected to an output of the output control unit for blocking abnormal signals when switching, a transform unit having an input connected to an output of the stabilizing circuit unit and an output connected to a load, the transform unit being configured to boost a voltage applied to the load, an intermittence unit for switchingly controlling a waveform of the voltage output from the transform unit, a feedback circuit unit for interrupting a current above a predetermined reference value, and a control unit for controlling the overall operations of the main body; a treatment electrode electrically connected to the main body, the treatment electrode being configured to contact a body part of a patient; a counter electrode pad electrically connected to the main body, the counter electrode being configured to contact another part of the patient's body; and an infrared lamp electrically connected to the main body.

Another aspect of the invention provides a high frequency electrotherapy apparatus for use in hair growth treatment, comprising: a first electrode comprising a ceramic layer; a second electrode; a voltage generator configured to generate a voltage between the first and second electrodes, the voltage having a frequency of about 250 kHz to about 330 kHz, the voltage having a waveform substantially free of a ripple component.

The voltage generator may comprise: a current generator configured to generate a high frequency current; and a transform unit having an input and an output, the input being connected to the current generator, the output being connected to the first and second electrodes, the transform unit being configured to adjust inductance so as to remove a ripple component from a high frequency current.

The voltage generator may further comprise: an intermittence unit having an output connected to an input of the transform unit, the intermittence unit being configured to adjust the waveform of the voltage. The voltage generator may further comprise: a switching controller having an output connected to an input of the intermittence unit; and a feedback circuit unit having an output and an input, the output being connected to an input of the switching controller, the input being connected to an output of the current generator. The switching controller may comprise a digital signal processor.

The apparatus may additionally comprise: a rectification unit configured to rectify a current supplied from an external power source; an output control unit configured to compare the rectified current with a reference voltage; and a stabilizing circuit unit having an input and an output, the input being connected to an output of the output control unit, the output being connected to an input of the transform unit. The first electrode may have a first surface configured to contact the body part, and the second electrode may have a second surface configured to contact the other body part, and the second surface may have an area about 20 times larger than the area of the first surface. The ceramic layer of the first electrode may be configured to substantially prevent a current flow between the first and second electrodes. The apparatus may further comprise an infrared lamp.

### BRIEF DESCRIPTION OF THE DRAWINGS

The above aspects and other features of various embodiments of the invention will become more apparent by describing in detail exemplary embodiments thereof with reference to the attached drawing figures, wherein;

FIG. 1 is a perspective view of a high-frequency electrotherapy apparatus according to an embodiment;

FIG. 2 is a block diagram illustrating a main body of an electrotherapy apparatus according to one embodiment;

FIG. 3 shows a structure of a treatment electrode of an electrotherapy apparatus according to one embodiment;

FIG. 4 shows a circuit diagram of an output control unit of the main body illustrated in FIG. 2;

FIG. 5 shows a circuit diagram of a feedback circuit unit of the main body illustrated in FIG. 2;

FIG. 6 shows circuit diagrams of an intermittence unit, a stabilizing circuit unit, and a transform unit of the main body illustrated in FIG. 2;

FIG. 7A shows a waveform of output voltage between electrodes, according to one embodiment;

FIG. 7B shows a conventional waveform of output voltage between electrodes;

FIG. 8A shows a circuit diagram of a display controller according to one embodiment; and

FIG. 8B shows a circuit diagram of a switching controller according to one embodiment.

### DETAILED DESCRIPTION OF CERTAIN INVENTIVE EMBODIMENTS

Hereinafter, certain embodiments will be described in detail with reference to the accompanying figures. In the following description, the same drawing reference numerals are used for the same elements even in different drawings.

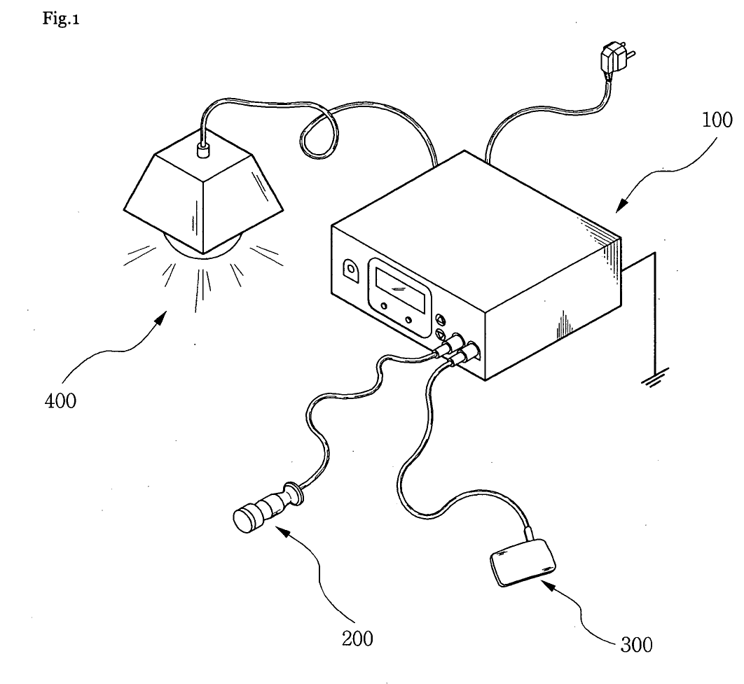

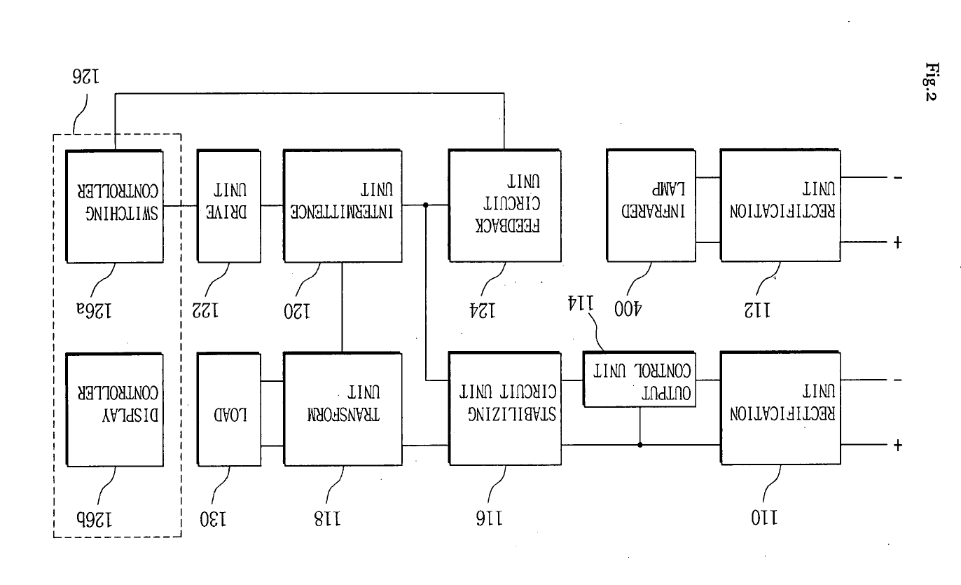

FIG. 1 is a perspective view showing a high-frequency electrotherapy apparatus according to an embodiment. FIG. 2 is a block diagram of the electrotherapy apparatus according to one embodiment.

As shown in FIGS. 1 and 2, the high-frequency electrotherapy apparatus comprises a main body 100 having a grounded frame, a treatment electrode 200 connected to the main body 100, a counter electrode pad 300, and an infrared lamp 400. 100331 Referring to FIG. 2, the main body 100 comprises rectification units 110 and 112, an output control unit 114, a stabilizing circuit unit 116, a transform unit 118, an intermittence unit 120, a drive unit 122, a feedback circuit unit 124, and a control unit 126. The control unit 126 comprises a switching controller 126a and a display controller 126b. In one embodiment, the frame of the main body 100 is made of metal.

The term “current generator” refers, in some embodiments, to the rectification unit 110. For other embodiments, the term “current generator” may refer together to the rectification unit 110, the output control unit 114, and the stabilizing circuit unit 116. The “current generator” may optionally further include the intermittence unit 120, the drive unit 122, and the feedback circuit unit 124. “Voltage generator” refers to the rectification unit 110, the output control unit 114, the stabilizing circuit unit 116, the transform unit 118, the intermittence unit 120, the drive unit 122, the feedback circuit unit 124, and the control unit 126. Thus, in some embodiments, the voltage generator may itself comprise a current generator.

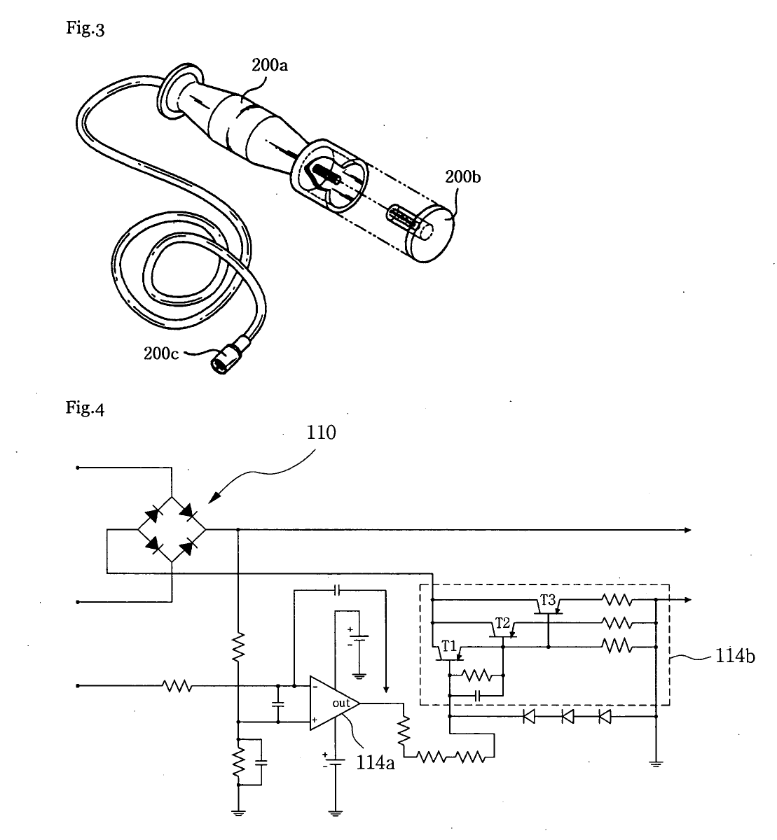

The treatment electrode 200, while being connected to the main body 100 through a signal wire, may contact a body part to be treated. As shown in FIG. 3, the treatment electrode 200 may include a shaft 200a, a treatment electrode pad 200b, and a terminal 200c. In some embodiments, the shaft 200a has a cylindrical form for a convenient grip, although other shapes are possible. The shaft 200a and the treatment electrode pad 200b are connected to each other, for example, by a screw. The treatment electrode pad 200b preferably comprises a metal disc coated with an insulating layer.

The counter electrode pad 300 may contact another body part to apply electricity, in combination with the treatment electrode pad 200. In one embodiment, a skin-contact area of the counter electrode pad 300 is approximately 20 times as large as a skin-contact area of the treatment electrode pad 200.

The infrared lamp 400 can be used to provide conditions appropriate for hair growth using the thermal effect of the infrared rays. Since it has a frequency within almost the same range as a natural frequency of molecules, the infrared ray promotes growth of hair when being projected onto a human skin.

The high-frequency electrotherapy apparatus according to the illustrated embodiment will now be described in greater detail. The main body 100 includes a power circuit and a controller circuit. Referring to FIG. 2, the main body 100 may include the rectification units 110 and 112 for decompressing and rectifying utility power, the output control part 114 connected to rear ends of the rectification units 110 and 112 to control current, the stabilizing circuit unit 116 connected to a rear end of the output control unit 114 to protect an element from abnormal signals when switching, the transform unit 118 having an input connected to the output of the stabilizing circuit for boosting voltage between the stabilizing circuit unit 116 and a load 130, the intermittence unit 120 switchingly controlling a waveform of the voltage output from the transform unit 118, the feedback circuit unit 124 for protecting the subject receiving electrotherapy, and the control unit 126 for controlling the overall operations of the main body 100.

In some embodiments, the control unit 126 includes the switching controller 126a and the display controller 126b which digitally perform precision control using a digital signal processor (DSP) and a microcontroller, respectively.

The frame of the main body 100 may be formed of a metal. Dangerous factors that may harm a human body can be avoided by grounding the metal frame.

FIG. 4 shows a circuit structure of the output control unit 114 of the high-frequency electrotherapy apparatus. As shown in FIG. 4, the output control unit 114 of the high-frequency electrotherapy apparatus can include a comparator 114a and a current value output unit 114b. The current value output unit 114b includes PNP transistors T1, T2 and T3 which are connected parallel to one another.

When a signal output from the display controller 126b is input to a negative input terminal of the comparator 114a, the signal is applied to a base B of the transistors T1, T2 and T3 of the current value output unit 114b through the comparator 114a. In other words, the amplitude of the signal output from the comparator 114a is determined by comparing amplitudes of the signals output from the rectification unit 110 and the display controller 126b. Collector current values (Ic) of the respective transistors T1, T2 and T3 are determined by the amplitude of the signal from the comparator 114a, thereby finally obtaining output current of a desired amplitude. The rectification unit 110 preferably comprises a bridge rectification circuit.

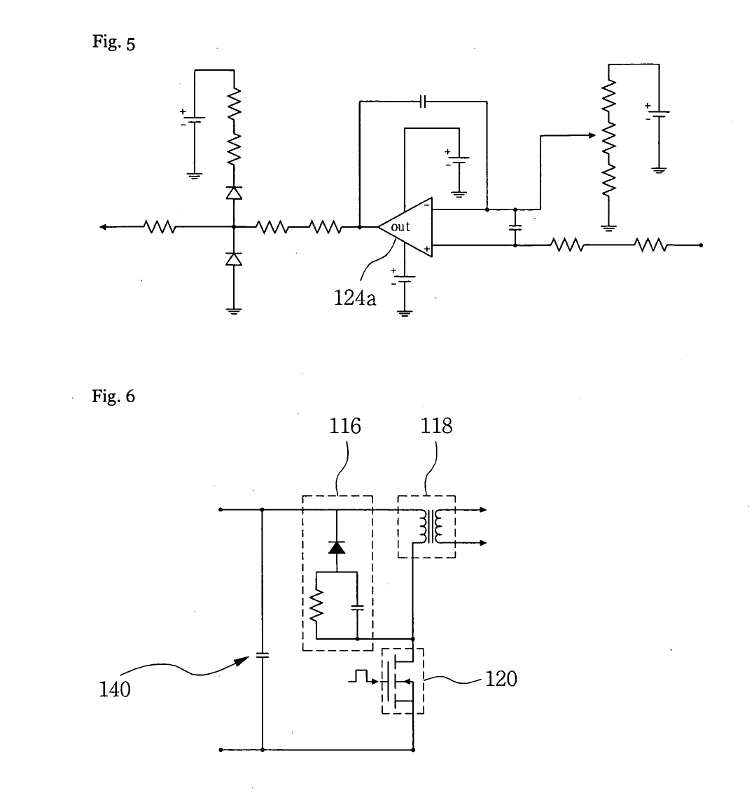

FIG. 5 illustrates the feedback circuit unit 124 of the high-frequency electrotherapy apparatus. The feedback circuit unit 124 prevents abnormal current from causing injury to the subject receiving the electrotherapy. As shown in FIG. 5, as a current is input to a positive input terminal of the comparator 124a, the comparator 124a compares the input current value with a reference current value, and outputs a control signal to the display controller 126b if the input current value is greater than the reference current value. Accordingly, the display controller 126b interrupts the signal and current being applied to the intermittence unit 120, thereby improving safety.

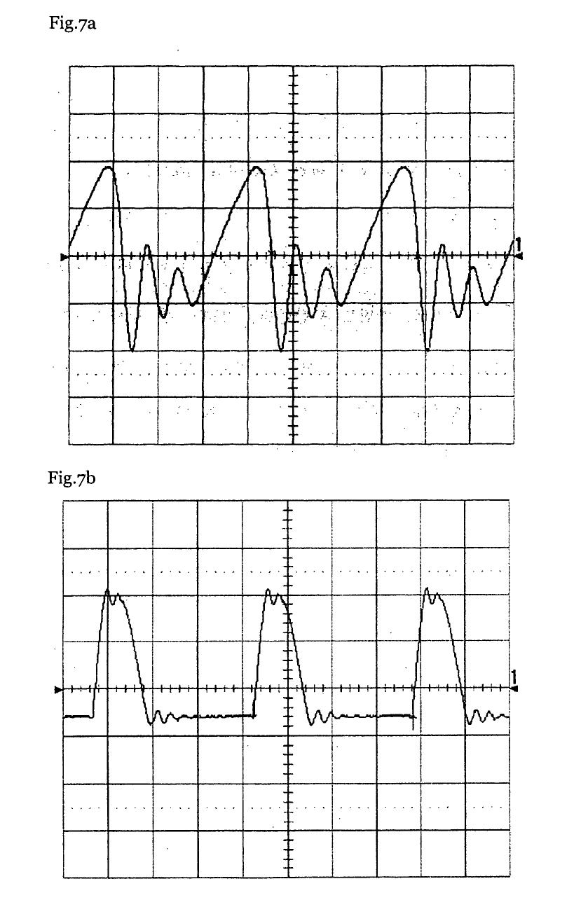

FIG. 6 illustrates the transform unit 118, the stabilizing circuit unit 116, and the intermittence unit 120 of the high-frequency electrotherapy apparatus. When a desired frequency and duty ratio are applied to the drive unit 122 from the switching controller 126a, the drive unit 122 outputs a switching signal of a range appropriate for switching of a metal-oxide semiconductor field effect transistor (MOSFET) which is part of the intermittence unit 120. The MOSFET is selectively switched according to the switching signal output from the drive unit 122, thereby obtaining desired frequency and waveform. According to this embodiment, a ripple voltage that can interfere with growth of hair can be removed by decreasing inductance L by reducing the number of turn of the transform unit 118. Also, a resonance point corresponds to that of a resonance capacitor 140.

By reducing the ripple voltage and the corresponding resonance point to the resonance capacitor 140, the waveform of voltage as shown in FIG. 7A can be obtained between the treatment electrode 200 and the counter electrode pad 300. FIG. 7B illustrates a waveform of a conventional voltage applied between electrodes. In FIG. 7B, the ripple components occurring between the electrodes considerably deteriorate functions for general beauty devices. In addition, the ripple components deteriorate the effect of promoting growth of hair. However, the ripple components are remarkably reduced in the waveform shown in FIG. 7A. Therefore, more electric energy can be transmitted to a second side of the transform unit 118, thereby improving the hair growth effect.

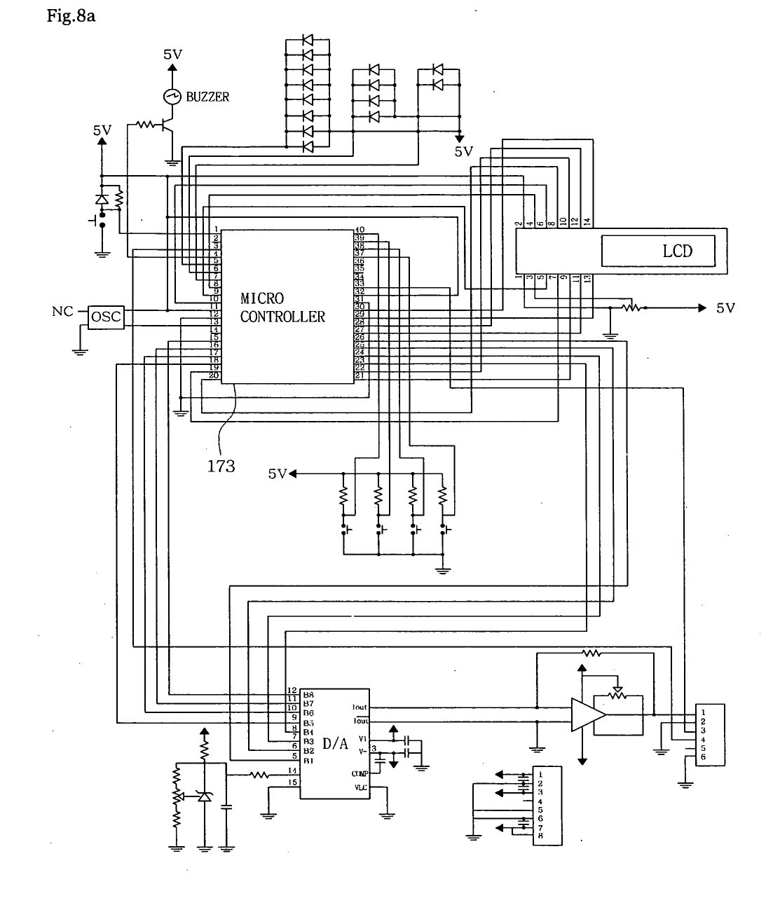

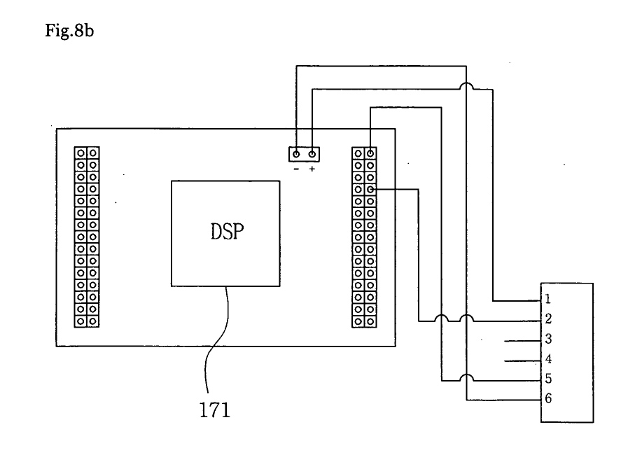

FIGS. 8A and 8B show the structure of the control unit 126 of the high-frequency electrotherapy apparatus according to one embodiment. More particularly, FIG. 8A shows the circuit structure of the display controller 126b. FIG. 8B illustrates the switching controller 126a.

As shown in FIG. 2, the control unit 126 of the high-frequency electrotherapy apparatus includes the switching controller 126a and the display controller 126b. The switching controller 126a measures and controls using a digital signal processor (DSP) 171, while the display controller 126b using a microcontroller 173. Here, the control unit 126 includes the switching controller 126a and the display controller 126b in order to prevent malfunction of the circuit caused by noise that may occur during the switching operation.

The microcontroller 173 performs a variety of functions, such as functions related to setting of operation time, output voltage and output energy, input of run/stop commands, over-current protection, and display. The DSP 171 performs high-speed switching.

As described above, the circuit of the high-frequency electrotherapy apparatus has a digital controlling system that performs measuring and controlling through the DSP 171 and the microcontroller 173. In a high-frequency electrotherapy apparatus to be used directly on a subject, such as a human body, such a digital controlling system helps improve safety for the human body, reduces uncomfortableness of a user, and generates and provides an optimal current for hair growth and a specific waveform appropriate for bioelectricity.

In other words, the high-frequency electrotherapy apparatus according to the embodiment is collectively operated and controlled by the DSP 170 and the microcontroller 173 to control the output voltage, frequency, current and waveform at the optimum condition. When any abnormality occurs, for example, abnormality in current, the main body 100 immediately stops its operations and raises an alarm. To this end, the high-frequency electrotherapy apparatus may further comprise an alarm.

Hereinafter, the operations of each component part of a high-frequency electrotherapy apparatus according to some embodiments will be described.

The main body 100 is connected to the treatment electrode 200 and the counter electrode pad 300 through one or more signal wires, and may also be connected to an infrared lamp 400 for maximum hair-growth effect. The infrared lamp 400 offers a beauty treatment suitable for the human skin using thermal effect of the infrared ray. This can be realized since the frequency of the infrared ray is within almost the same range as natural frequency of molecules. When the infrared ray is irradiated onto a body part, thereby generating electroresonance, energy of the infrared wave appropriate for the beauty treatment can be absorbed into the body part efficiently. The optimum beauty treatment conditions can be accomplished by emitting the thermal energy in this state. Emission of the thermal energy induces somatological heat energy by the high-frequency current. The somatological heat energy has temperature of approximately about 42 to about 46° C. The maximum diathermancy of the somatological heat energy is approximately 2 mm into a deep part.

Infrared rays from the infrared lamp 400 may be projected onto the body part requiring the treatment, independently from the high-frequency therapy using the treatment electrode 200 and the counter electrode pad 300. Alternatively, the infrared lamp 400 may be projected before, during, or after the high-frequency therapy to promote effect of the treatment.

The treatment electrode 200 is contacted directly with the body part to be treated whereas the counter electrode pad 300 is contacted with the body for electric conduction, as a couple with the treatment electrode 200. About 1.0 to about 1.5 kV of AC output voltage and about 250 to about 330 kHz of frequency are applied to the treatment electrode 200. An average input current of a first side is approximately 0.2 to 0.7 A, optionally 0.5 A. Capacitance transformation is applied as the electric system.

The treatment electrode 200 may be formed by coating a circular metal disc with a semiconductive ceramic layer having an excellent mechanical strength and preferably having no pin holes, so as to improve safety for the subject and to facilitate manufacturing of the electrodes. Since the treatment electrode 200 is partially insulated, a current does not directly flow through the subject, for example, through the human body. Accordingly, the current does not do any harm to the subject.

In conventional electrotherapy, a treatment electrode may comprise glass or general metal. Sodium (Na) from excretion such as sweat of the human body may react with the glass of the treatment electrode, which may cause explosion. Furthermore, when the metal is heated to a high temperature, the subject may be burned. By using a treatment electrode 200 as described herein, such dangerous aspects of the conventional treatment electrode can be excluded.

In the treatment electrode 200, an alternating capacitive coupling is accomplished between the counter electrode pad 300 and the skin by the high-frequency waveform of approximately 250 to 330 kHz, so that a current flows through the subject's body. Because the human skin is covered with an almost nonconductive horny layer, the high-frequency AC needs to be projected to influence even deep tissue under an inner skin layer, passing through an outer skin layer. In this regard, depth effect should be maintained optimally without damaging the human body or skin.

In this embodiment, the high-frequency current of approximately 250 kHz to 330 kHz is modulated to have an optimum waveform for maintaining the optimum depth effect and achieving the maximum thermal effect. The thermal effect is generated at the deep tissue contacted with the treatment electrode 200 having relatively higher current density.

The somatological heat generated by the thermal effect can be applied for various treatments. For example, when the electrical stimulation is applied onto an acupuncture point where the electric energy is most conductive, metabolism is highly enhanced. The somatological heat may also be applied for treatments by renewal and activation of cells. The same principle is used in acupuncture and moxibustion belonging to oriental medicine. This principle is closely related to western medicine as well.

A method for utilizing the high-frequency electrotherapy apparatus according to the embodiment will now be explained.

Upon application of power to the high-frequency electrotherapy apparatus, a high frequency of approximately 250 kHz to 330 kHz is generated in the main body 100. A dedicated cream having high conductivity is applied on the body part to be treated. Preferably, the subject is human, although treatment of other subjects is possible as will be apparent to the skilled artisan. As the treatment electrode 200 is attached to the body part, a current is conducted toward the counter electrode pad 300 attached to another part of the subject's body. Accordingly, the somatological heat is generated by the electric energy and resistance of the body. Preferably, the subject is human, although treatment of other subjects is possible as will be apparent to the skilled artisan.

The thermal effect occurring in the tissue is caused by influences of the current and an electromagnetic field, in accordance with Joule's law. Therefore, sum of the thermal energy generated in tissue is proportional to a square of the average current passed through the tissue, resistance of the tissue, and treatment time.

Although a low-voltage current and a high-voltage pulsation current having a relatively low average current are not able to achieve desired thermal effect, the high-frequency electrotherapy apparatus capable of diathermy therapy using the high-frequency current is satisfactory with regard to the thermal effect. Generation of heat is influenced by a position of the electrode and intensity of the current. The thermal effect is especially high in the skin and subcutaneous fat under the electrode, having relatively higher resistance. However, in blood and muscle having low resistance and high conductivity, the thermal effect is relatively poor.

The thermal effect in the tissue of the human body causes expansion of blood vessels, thereby increasing blood flux in capillary vessels. More specifically, when temperature of a topical part of the tissue increases more than 42° C., the vessels are expanded, hydrostatic pressure in the capillary vessels are increased, and the blood flow increases by about 4 to 5 times as much as usual. The generated somatological heat energy is applied to the treatment electrode 200, by radiating heat outward. The electric energy treats insides of the human body such as cells, muscle and blood by the electric current. The treatment electrode 200 in contact with the human skin stimulates the acupuncture point, bringing the same effects as massage and acupuncture. Thus, both inside and outside of the human body are cured.

As explained above, the high-frequency electrotherapy apparatus has the following effects.

The apparatus can convert the high-frequency electric energy of a specific level into the somatological heat energy by conducting the electric energy deeply into the human body. Thus-generated somatological heat energy restrains aging, elongates an aging period, enhances self-healing power, helps energy generation and cell renewal, promotes blood purification and circulation, and promotes excretion of body wastes.

Molecular motion deteriorates at a body part having lesion due to lack of natural wave. Therefore, the somatological energy decreases, thereby deteriorating the cell renewal function due to poor metabolism and malfunction of endocrine gland. Vibration of energy-generating wave stimulates the molecular motion and activates the metabolism, thereby improving functions of neurons of capillary vessels and cell membrane, and also improving renewal of tissues. Such processes help fundamentally cure various diseases.

Promotion of blood purification and blood circulation causes dissociation of electrolytes of blood through infiltration of the high-frequency current into the tissue, and encourages cell movement by increasing and balancing ions. Permeability of cell membrane is encouraged and oxygen consumption is increased through chemical reaction of K+ and Na+ required for metabolism. Mutual infiltration between Ca salt and Mg salt stimulates an autonomic nerve to help contraction and expansion of vessels, thereby promoting blood circulation. Demodex which affects growth of hair, among a great number of parasites, especially, can be easily eliminated using the thermal effect and intensity of the electric filed, generated by high voltage and high frequency. The high frequency promotes vibration of H+ ions in the human body and mutual infiltration of intracellular and extracellular fluids. As a result, greasy materials that block the blood vessels are dissolved and excreted to the outside, thereby improving blood purification. Also, blood circulation is highly promoted by expanding capillary vessels, so that the effect of stimulating hair growth can be maximized.

While the invention has been shown and described with reference to certain embodiments thereof, it will be understood by those skilled in the art that various changes in form and details may be made therein without departing from the spirit and scope of the invention as defined by the appended claims.

What is claimed is:

1. A high-frequency electrotherapy apparatus comprising:
a main body comprising:
a current generator for generating a high frequency current;
a control unit for controlling generation of a high frequency current, and
a grounded frame;
a treatment electrode electrically connected to the main body, the treatment electrode being configured to contact a body part of a patient;
a counter electrode pad electrically connected to the main body, the counter electrode being configured to contact another body part of the patient; and
an infrared lamp electrically connected to the main body.

2. The apparatus of claim 1, wherein the high-frequency current has a voltage of about 1.0 to about 1.5 kV, a frequency of about 250 to about 330 kHz, and an average input current of about 0.2 to about 0.7 A.

3. The apparatus of claim 1, wherein the current generator comprises:
a rectification unit for rectifying power supply;
an output control unit connected to an output of the rectification unit for controlling a current output from the rectification unit; and
a stabilizing circuit unit connected to an output of the output control unit for blocking abnormal signals when switching,
and wherein the main body additionally comprises:
a transform unit having an input connected to an output of the stabilizing circuit unit and an output connected to a load, the transform unit being configured to boost a voltage applied to the load;
an intermittence unit for switchingly controlling a waveform of the voltage output from the transform unit; and
a feedback circuit unit for preventing a current above a reference value.

4. The apparatus of claim 1, wherein the control unit comprises:
a switching controller for measuring and controlling through a digital signal processor (DSP); and
a display controller for controlling setting of output voltage and output energy, input of run/stop commands, cover-current protection, and display, through a microcontroller.

5. The apparatus of claim 3, wherein, when current above the reference value is detected by the feedback circuit unit, the control unit interrupts application of a signal and current to the intermittence unit.

6. The apparatus of claim 1, wherein the treatment electrode comprises:
a cylindrical shaft;
a terminal connected to the shaft; and
a thin ceramic layer coating the terminal.

7. The apparatus of claim 1, wherein a skin-contact area of the counter electrode pad is approximately 20 times as large as a skin-contact area of the treatment electrode pad.

8. The apparatus of claim 3, wherein the intermittence unit switchingly controls a waveform of the voltage output from the transform unit to have an optimum pulse shape for maximizing a hair growth effect.

9. The apparatus of claim 3, wherein the transform unit is configured to adjust inductance so as to remove a ripple voltage generated between the electrodes.

10. The apparatus of claim 1, wherein the infrared lamp is configured to project infrared rays onto the body part.

11. A high-frequency electrotherapy apparatus comprising:
a main body comprising:
a grounded frame,
a rectification unit for rectifying power supply,
an output control part connected to an output of the rectification unit for controlling a current output from the rectification unit,
a stabilizing circuit unit connected to an output of the output control unit for blocking abnormal signals when switching,
a transform unit having an input connected to an output of the stabilizing circuit unit and an output connected to a load, the transform unit being configured to boost a voltage applied to the load,
an intermittence unit for switchingly controlling a waveform of the voltage output from the transform unit,
a feedback circuit unit for interrupting a current above a predetermined reference value, and
a control unit for controlling the overall operations of the main body;
a treatment electrode electrically connected to the main body, the treatment electrode being configured to contact a body part of a patient;
a counter electrode pad electrically connected to the main body, the counter electrode being configured to contact another part of the patient's body; and
an infrared lamp electrically connected to the main body.

12. A high frequency electrotherapy apparatus for use in hair growth treatment, comprising:
a first electrode comprising a ceramic layer;
a second electrode;
a voltage generator configured to generate a voltage between the first and second electrodes, the voltage having a frequency of about 250 kHz to about 330 kHz, the voltage having a waveform substantially free of a ripple component.

13. The apparatus of claim 12, wherein the voltage generator comprises:
a current generator configured to generate a high frequency current; and
a transform unit having an input and an output, the input being connected to the current generator, the output being connected to the first and second electrodes, the transform unit being configured to adjust inductance so as to remove a ripple component from a high frequency current.

14. The apparatus of claim 13, wherein the voltage generator further comprises:
an intermittence unit having an output connected to an input of the transform unit, the intermittence unit being configured to adjust the waveform of the voltage.

15. The apparatus of claim 14, wherein the voltage generator further comprises:
a switching controller having an output connected to an input of the intermittence unit; and
a feedback circuit unit having an output and an input, the output being connected to an input of the switching controller, the input being connected to an output of the current generator.

16. The apparatus of claim 15, wherein the switching controller comprises a digital signal processor.

17. The apparatus of claim 15, additionally comprising:
a rectification unit configured to rectify a current supplied from an external power source;
an output control unit configured to compare the rectified current with a reference voltage; and
a stabilizing circuit unit having an input and an output, the input being connected to an output of the output control unit, the output being connected to an input of the transform unit.

18. The apparatus of claim 12, wherein the first electrode has a first surface configured to contact the body part, and the second electrode has a second surface configured to contact the other body part, and wherein the second surface has an area about 20 times larger than the area of the first surface.

19. The apparatus of claim 12, wherein the ceramic layer of the first electrode is configured to substantially prevent a current flow between the first and second electrodes.

20. The apparatus of claim 12, further comprising an infrared lamp.
